(19) United States
(12) Patent Application Publication
(10) Pub. No.: US 2013/0110001 A1
(43) Pub. Date: May 2, 2013

(54) GUIDEWIRE

(71) Applicant: ASAHI INTECC CO., LTD., Nagoya-shi (JP)

(72) Inventors: Naohiko MIYATA, Nagoya-shi (JP); Hirokatsu TAKEMOTO, Seto-shi (JP)

(73) Assignee: ASAHI INTECC CO., LTD., Nagoya-shi (JP)

(21) Appl. No.: 13/657,258

(22) Filed: Oct. 22, 2012

(30) Foreign Application Priority Data
Oct. 31, 2011 (JP) 2011-238568

(51) Int. Cl. A61M 25/09

(52) U.S. Cl. 600/585

(57) ABSTRACT

A guidewire includes a core wire, a coil body that covers the core wire, a tip portion that fixes a distal end of the core wire to a distal end of the coil body, and a resin layer formed on an outer peripheral surface of the core wire. The resin layer is formed at least on an outer peripheral surface of a distal end portion of the core wire so as to extend toward a proximal end of the core wire from a position separated from the tip portion toward a proximal end of the guidewire.

### CROSS-REFERENCE TO RELATED APPLICATIONS

This application claims priority to Japanese Patent Application No. 2011-238568 filed in the Japan Patent Office on Oct. 31, 2011, the entire contents of which are incorporated herein by reference.

### BACKGROUND

The disclosed embodiments of the present invention relate to a guidewire suitable for use in the medical field. Medical guidewires are medical devices that are used as guides for inserting a catheter into, for example, a blood vessel, a ureter, or an organ, or inserting an indwelling device into an aneurysm of a blood vessel.

A related art guidewire includes a tapered coil body provided with resin portions on the inner and outer sides thereof (see, for example, Japanese Unexamined Patent Application Publication No. 2010-268888). In addition, another related art guidewire has a double-coil-body structure which includes a core wire fixed to a plurality of coil bodies by a fixing portion and in which the coil bodies exist on both the distal-end side and proximal-end side of the fixing portion (see, for example, U.S. Pat. No. 5,345,945).

### SUMMARY

However, in the guidewire according to Japanese Unexamined Patent Application Publication No. 2010-268888, the inner space of a coil spring is provided in a distal end portion of the guidewire and is filled with a resin portion, and the resin portion is connected to a tip portion of the guidewire. Therefore, although the torque transmission performance of the distal end portion of the guidewire and supportability that affects the insertability of a medical device such as a balloon catheter or a stent may be increased, there is a problem that the flexural rigidity of the distal end portion is excessively high and sufficient flexibility cannot be obtained. Further, when the flexural rigidity of the distal end portion is excessively high, ease of shape changing of a portion of the coil body near the tip portion of the guidewire to a curved or bent shape is reduced.

Moreover, the guidewire disclosed in U.S. Pat. No. 5,345,945 has a double-coil-body structure. Therefore, the rigidity of a portion of the guidewire that is closer to the distal end thereof than the fixing portion which fixes the core wire to the coil bodies is increased, and the torque transmission performance and supportability are increased accordingly. However, there is a problem that the rigidity of a portion of the guidewire that is closer to the proximal end thereof than the fixing portion cannot be increased by the double-coil-body structure and therefore sufficient supportability cannot be obtained.

The disclosed embodiments of the present invention have been made in view of the above-described problems. Therefore, an object of the disclosed embodiments of the present invention is to provide a guidewire in which supportability of a distal end portion of the guidewire is increased and good flexibility is ensured so that ease of shaping is increased.

According to an aspect of the present invention, a guidewire includes a core wire, a coil body that covers the core wire, a tip portion that fixes a distal end of the core wire and a distal end of the coil body to each other, and a resin layer formed on an outer peripheral surface of the core wire. The resin layer is formed at least on an outer peripheral surface of a distal end portion of the core wire so as to extend toward a proximal end of the core wire from a position separated from the tip portion toward a proximal end of the guidewire.

With this structure, since the resin layer is formed at least on the outer peripheral surface of the distal end portion of the core wire, the distal end portion of the guidewire according to an embodiment of the present invention has sufficient supportability. In addition, since the resin layer is separated from the tip portion toward the proximal end of the guidewire, the distal end portion of the guidewire according to an embodiment of the present invention has sufficient flexibility, and ease of shaping thereof is sufficiently ensured.

According to an embodiment of the guidewire of the present invention, supportability of the distal end portion is increased and good flexibility is ensured so that ease of shaping is also increased. The preceding, as well as a number of additional features will now be described with reference to the following drawings, in which:

### BRIEF DESCRIPTION OF THE DRAWINGS

FIG. 1 illustrates a guidewire according to a first embodiment.

FIG. 2A is a vertical sectional view of a cylindrical body made of resin.

FIG. 2B is a sectional view of FIG. 2A taken along line IIB-IIB.

FIG. 3 illustrates a guidewire according to a second embodiment.

FIG. 4 illustrates a guidewire according to a third embodiment.

FIG. 5 illustrates a guidewire according to a fourth embodiment.

FIG. 6 illustrates a guidewire according to a fifth embodiment.

FIG. 7 illustrates a guidewire according to a sixth embodiment.

### DETAILED DESCRIPTION OF EMBODIMENTS

Guidewires according to embodiments of the present invention will now be described.

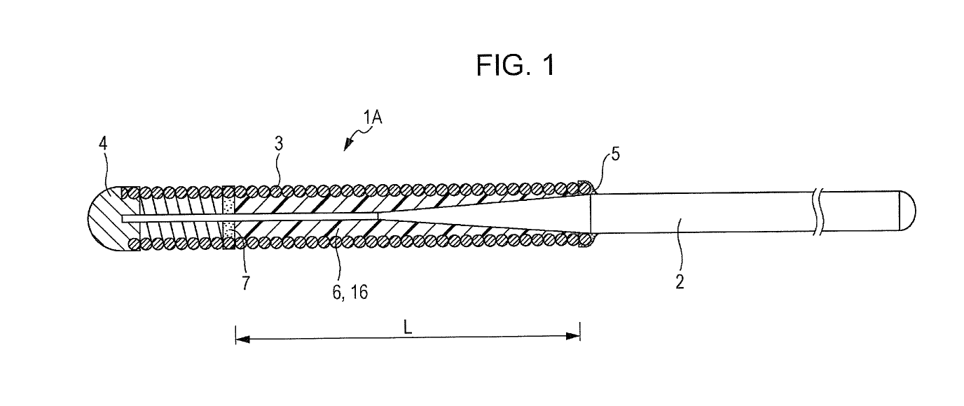

Referring to FIG. 1, a guidewire 1A, which is used for medical purposes, includes a round-bar-shaped core wire 2 made of a metal material. The core wire 2 is tapered such that the diameter thereof is smaller at the distal end of the core wire 2 than at the proximal end of the core wire. As used herein, the term “proximal end” means the portion of the guidewire 1A that is closer to a user, while the guidewire 1A is in use. As used herein, the term “distal end” means the portion of the guidewire 1A that is farther from a user, while the guidewire 1A is in use.

A coil body 3 made of a metal material is arranged so as to cover a distal end portion of the core wire 2. A substantially hemispherical tip portion 4 is formed so as to fix the distal end of the core wire 2 and the distal end of the coil body 3 together. The proximal end of the coil body 3 is fixed to the core wire 2 by a first fixing portion 5.

A resin layer 6 is formed at least on the outer peripheral surface of the distal end portion of the core wire 2 and inside the coil body 3. That is, between the core wire 2 and the coil body 3. The resin layer 6 extends toward the proximal end of the guidewire 1A from a position separated from the tip portion 4 toward the proximal end of the core wire 2. More specifically, the distal end of the resin layer 6 is in contact with a second fixing portion 7, which is separated from the tip portion 4 toward the proximal end of the guidewire 1A, and the proximal end of the resin layer 6 is in contact with the first fixing portion 5. The resin layer 6 is in contact with the inner peripheral surface of the coil body 3 over the entire length L thereof in the axial direction of the core wire 2.

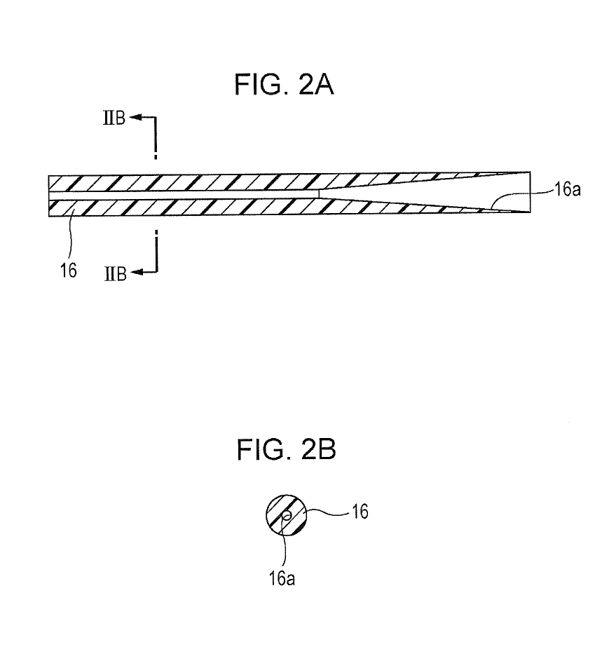

The resin layer 6 includes a cylindrical body 16 made of resin, as illustrated in FIG. 2, and is easily formed by inserting the core wire 2 into a cylinder hole 16a in the cylindrical body 16. The dimensions and shapes of the cylindrical body 16 and the cylinder hole 16a desirably correspond to the external shape of a portion of the core wire 2 on which the resin layer 6 is formed. Polyurethane, polyamide, polyimide, and various elastomers, for example, are suitable as the material of the cylindrical body 16. In particular, the cylindrical body 16 is preferably made of polyimide since the flexural rigidity and supportability can be increased.

The first fixing portion 5 and the second fixing portion 7 may be formed by, for example, adhesion, soldering, or welding.

In the guidewire 1A having the above-described structure, the resin layer 6 is formed on the outer peripheral surface of the core wire 2. Therefore, the distal end portion of the guidewire 1A has sufficient supportability. The distal end of the resin layer 6 may be separated from the tip portion 4. That is, in one embodiment, the resin layer 6 may extend axially from a first fixing portion 5 closer to the proximal end of the guidewire 1A toward a distal end of the guidewire 1A, but the resin layer 6 need not reach all the way to the tip portion 4. Therefore, the distal end portion of the guidewire 1A has sufficient flexibility, and ease of shaping thereof is sufficiently ensured.

In addition, since the resin layer 6 is interposed between the coil body 3 and the core wire 2 while being in contact therewith, supportability of the distal end portion of the guidewire 1A is further increased. It is not necessary that the resin layer 6 be in contact with the inner peripheral surface of the coil body 3 over the entire length L thereof. In fact, in one embodiment the resin layer 6 may be partially in contact with the inner peripheral surface of the coil body 3. For example, the resin layer 6 may include a contact portion that is in contact with the inner peripheral surface of the coil body 3 and a portion that is disposed on the distal-end side of the contact portion and that is not in contact with the inner peripheral surface of the coil body 3. In the case where the resin layer 6 includes the portion that is not in contact with the inner peripheral surface of the coil body 3 on the distal-end side of the contact portion, the resin layer 6 may be formed such that the outer diameter thereof decreases toward the distal end thereof. In such a case, the flexibility can be further increased while good supportability of the distal end portion of the resin layer 6 is ensured.

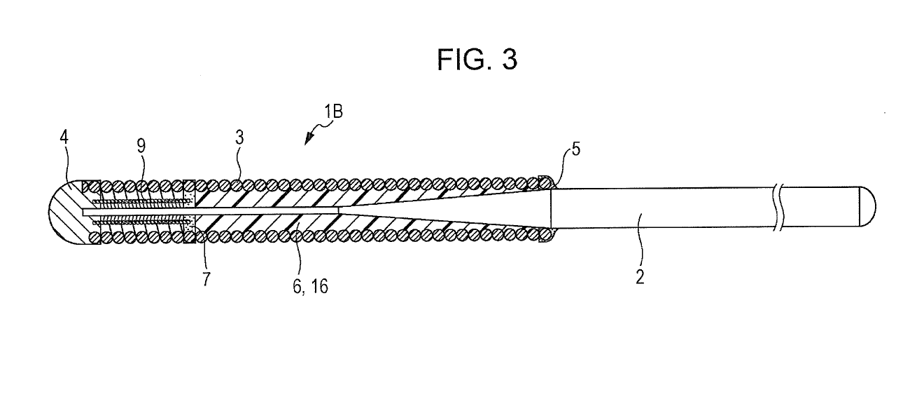

A guidewire 1B according to a second embodiment will now be described with reference to FIG. 3. Components similar to those of the first embodiment are denoted by the same reference numerals, and explanations thereof are thus omitted.

As illustrated in FIG. 3, the guidewire 1B includes an inner coil body 9 that is disposed inside the coil body 3 and covers the distal end portion of the core wire 2. More specifically, the distal end of the inner coil body 9 is fixed to the tip portion 4, and the proximal end of the inner coil body 9 is connected to the distal end of the resin layer 6 with the second fixing portion 7, which is formed at the distal end of the resin layer 6, interposed therebetween. The inner coil body 9 is formed of a multiple-wire coil obtained by twisting a plurality of coil wires together. Alternatively, the inner coil body 9 may be formed of a single-wire coil.

When the inner coil body 9 is disposed between the tip portion 4 and the resin layer 6, supportability of a portion of the guidewire 1B around the tip portion 4 can be increased while good flexibility of the distal end portion of the guidewire 1B is ensured. Since the inner coil body 9 is provided between the distal end of the resin layer 6 and the tip portion 4, a rigidity gap at the distal end of the resin layer 6 can be reduced and supportability of the guidewire 1B can be balanced from the proximal end to the distal end thereof.

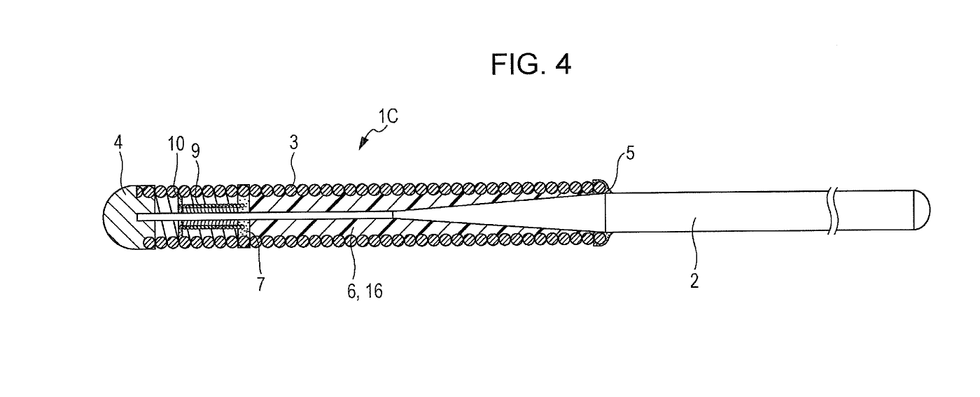

A guidewire 1C according to a third embodiment will now be described with reference to FIG. 4. Components similar to those of the first and second embodiments are denoted by the same reference numerals, and explanations thereof are thus omitted.

As illustrated in FIG. 4, the guidewire 1C includes an inner coil body 9 that is disposed between the tip portion 4 and the resin layer 6. The distal end of the inner coil body 9 is separated from the tip portion 4 toward the proximal end of the guidewire 1C, and is connected to a third fixing portion 10, which is provided between the tip portion 4 and the resin layer 6. The proximal end of the inner coil body 9 is connected to the resin layer 6 with the second fixing portion 7 interposed therebetween.

Since the inner coil body 9 is connected to the resin layer 6 with the second fixing portion 7 interposed therebetween, the rigidity gap between the inner coil body 9 and the resin layer 6 is reduced and supportability of the guidewire 1C can be balanced from the proximal end to the distal end thereof. In addition, since the distal end of the inner coil body 9 is separated from the tip portion 4 toward the proximal end of the guidewire 1C, the distal end portion of the guidewire 1C has sufficient flexibility.

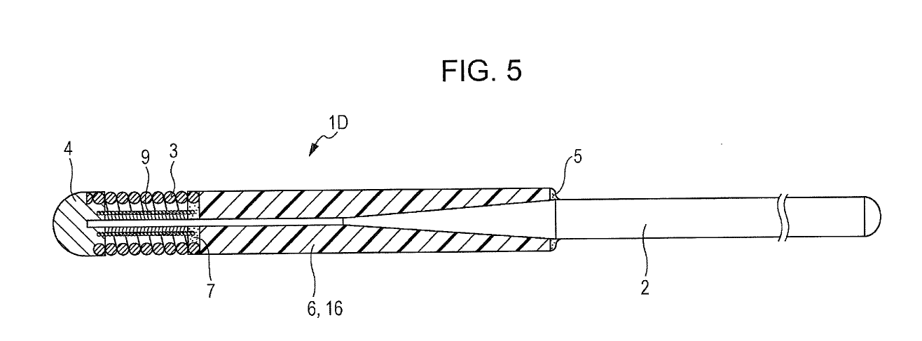

A guidewire 1D according to a fourth embodiment will now be described with reference to FIG. 5. Components similar to those of the first to third embodiments are denoted by the same reference numerals, and explanations thereof are thus omitted.

As illustrated in FIG. 5, the resin layer 6 is formed at least on the outer peripheral surface of the distal end portion of the core wire 2 and on the proximal-end side of the coil body 3. More specifically, the proximal end of the coil body 3 is in contact with the distal end of the resin layer 6 with the second fixing portion 7 interposed therebetween. The inner coil body 9 is disposed between the tip portion 4 and the distal end of the resin layer 6.

Also in this structure, the distal end portion of the guidewire 1D has good supportability owing to the resin layer 6. In addition, since the distal end of the resin layer 6 is separated from the tip portion 4 toward the proximal end of the guidewire 1D, the distal end portion of the guidewire 1D has good flexibility and ease of shaping thereof can be increased. In addition, since the inner coil body 9 is provided, the rigidity gap between the tip portion 4 and the resin layer 6 is appropriately reduced and supportability of the guidewire 1D can be balanced from the proximal end to the distal end thereof.

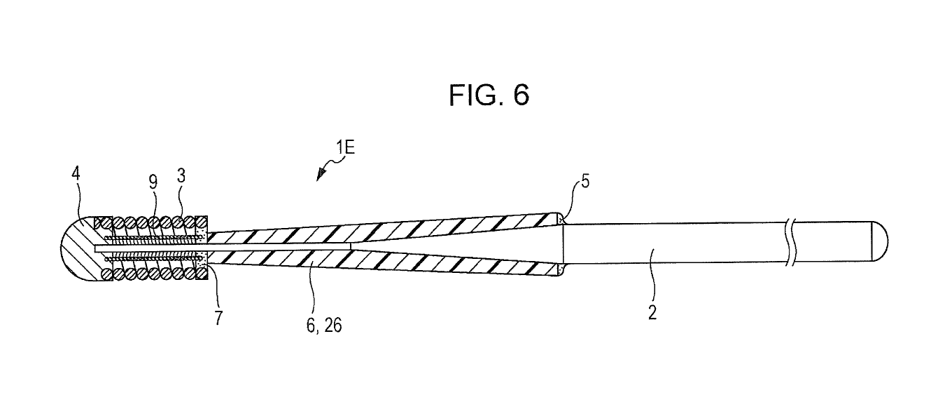

A guidewire 1E according to a fifth embodiment will now be described with reference to FIG. 6. Components similar to those of the first to fourth embodiments are denoted by the same reference numerals, and explanations thereof are thus omitted.

A part of the guidewire 1E illustrated in FIG. 6 that corresponds to the resin layer 6 has an outer diameter that increases from a distal end of the core wire 2 toward the proximal end of the core wire 2.

With this structure, supportability of the resin layer 6 increases toward the proximal end of the guidewire 1E. Accordingly, a medical device, such as a balloon catheter or a stent, can be smoothly inserted along the guidewire 1E. The resin layer 6 is preferably simply formed of a cylindrical body 26 made of resin which has a diameter that increases from a distal end of the guidewire 1E toward the proximal end of the guidewire 1E.

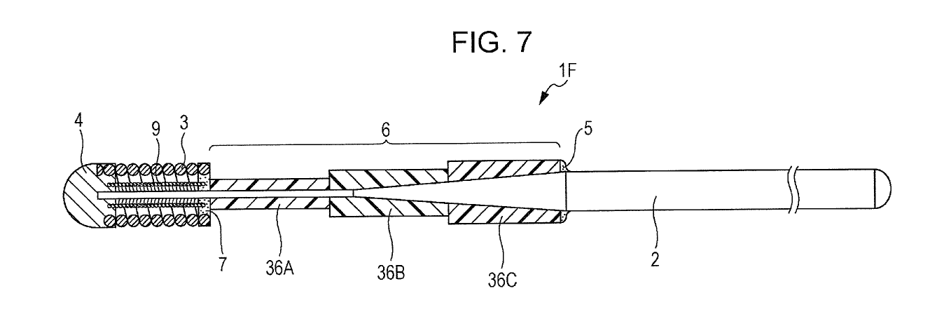

A guidewire 1F according to a sixth embodiment will now be described with reference to FIG. 7. Components similar to those of the first to fifth embodiments are denoted by the same reference numerals, and explanations thereof are thus omitted.

A part of the guidewire 1F illustrated in FIG. 7 that corresponds to the resin layer 6 has an outer diameter that increases stepwise toward the proximal end of the core wire 2. For instance, in one embodiment the resin layer 6 may include three independent cylindrical bodies 36A to 36C made of resin that have different outer diameters. The resin layer 6 is formed by fitting the cylindrical bodies 36A to 36C onto the core wire 2 in descending order of outer diameter. In other embodiments, the cylindrical bodies 36A to 36C need not be limited to three. For example, in another embodiment, the resin layer 6 may instead be formed of a single cylindrical body made of resin that has an outer diameter that changes stepwise.

With this structure, the resin layer 6 having the rigidity that gradually changes can be easily formed simply by inserting the core wire 2 through the cylindrical bodies 36A to 36C.

The embodiments of the present invention are not limited to the above-described first to sixth embodiments. In fact, various design changes may be made and the embodiments may be combined as appropriate without departing from the scope of the present invention. For example, the resin layer 6 may be formed by applying a resin material in a molten state to the outer peripheral surface of the core wire 2 and curing the resin material. In this case, when the proximal end of the inner coil body 9 is to be connected to the distal end of the resin layer 6, the proximal end of the inner coil body 9 may be inserted into the resin layer 6 and be set to the embedded state in the curing process. In the second to sixth embodiments, the proximal end of the inner coil body 9 is connected to the resin layer 6 with the second fixing portion 7 interposed therebetween from the viewpoint of productivity. However, the proximal end of the inner coil body 9 may instead be connected to the distal end of the resin layer 6 by, for example, bringing the proximal end of the inner coil body 9 into contact with the distal end of the resin layer 6 without providing the second fixing portion 7 therebetween. In addition, in the fourth to sixth embodiments, the inner coil body 9 may be omitted.

While the foregoing embodiments have been shown and described in detail, the foregoing description is in all aspects illustrative and not restrictive. It is therefore understood that numerous modifications and variations can be devised without departing from the spirit and scope of the invention.

What is claimed is:

1. A guidewire comprising:
a core wire;
a coil body that covers the core wire;
a tip portion that fixes a distal end of the core wire to a distal end of the coil body; and
a resin layer formed on an outer peripheral surface of the core wire, wherein
the resin layer is formed at least on an outer peripheral surface of a distal end portion of the core wire so as to extend toward a proximal end of the core wire from a position separated from the tip portion toward a proximal end of the guidewire.

2. The guidewire according to claim 1, wherein the resin layer is arranged at least inside the coil body, and at least a part of an inner peripheral surface of the coil body is in contact with the resin layer.

3. The guidewire according to claim 1, further comprising:
an inner coil body that is disposed inside the coil body and covers the core wire, wherein
a distal end of the inner coil body is fixed to the tip portion, and
a proximal end of the inner coil body is connected to at least a distal end of the resin layer.

4. The guidewire according to claim 1, further comprising:
an inner coil body that is disposed inside the coil body and covers the core wire, wherein
a distal end of the inner coil body is separated from the tip portion toward the proximal end of the guidewire, and
a proximal end of the inner coil body is connected to at least a distal end of the resin layer.

5. The guidewire according to claim 1, wherein the resin layer is formed of a cylindrical body made of resin.

6. The guidewire according to claim 1, wherein a part of the guidewire that corresponds to the resin layer has an outer diameter that increases toward the proximal end of the core wire.

7. The guidewire according to claim 5, wherein the cylindrical body that forms the resin layer has an outer diameter that increases stepwise toward the proximal end of the core wire.

8. A guidewire comprising:
a core wire having a distal end and a proximal end;
a coil body that covers at least part of the core wire;
a tip portion that fixes the distal end of the core wire to the coil body;
a resin layer formed around at least part of the core wire;
a first fixing portion that joins the proximal end of the coil body to the core wire; and
a second fixing portion that is closer to the tip portion than the first fixing portion, the second fixing portion joining the core wire to the coil body, wherein
the resin layer extends axially only from the first fixing portion to the second fixing portion.

9. The guidewire according to claim 8, further comprising:
an inner coil body disposed between the core wire and the coil body.

10. The guidewire according to claim 9, wherein the inner coil body has a first end coupled to the second fixing portion and a second end coupled to the tip portion.

11. The guidewire according to claim 9, further comprising:
a third fixing portion that is closer to the tip portion than the second fixing portion, the third fixing portion joining the core wire to the coil body, wherein
the inner coil body has a first end coupled to the second fixing portion and a second end coupled to the third fixing portion.

12. The guidewire according to claim 9, wherein the inner coil body overlaps only a portion of the core wire, such that a portion of the core wire spans through the third fixing portion to be coupled to the tip portion.

13. The guidewire according to claim 9, wherein the coil body does not overlap the core wire in a region of the guidewire between the first fixing portion and the second fixing portion.

14. The guidewire according to claim 9, wherein an outer diameter of the guidewire in a region of the guidewire between the tip portion and the second fixing portion is larger than an outer diameter of the guidewire in a region of the guidewire between the second fixing portion and the first fixing portion.

15. The guidewire according to claim 9, wherein the resin portion includes a plurality of individual cylindrical bodies, each individual cylindrical body having a diameter that decreases from the first fixing portion toward the second fixing portion.
